(19) United States
(12) Patent Application Publication
(10) Pub. No.: US 2006/0004415 A1
(43) Pub. Date: Jan. 5, 2006

(54) Defibrillator with improved output stage

(76) Inventor: Heinz Bucher, Rottweil (DE)

Correspondence Address: PAULEY PETERSEN & ERICKSON, 2800 WEST HIGGINS ROAD, SUITE 365, HOFFMAN ESTATES, IL, 60195, US

(21) Appl. No.: 10/535,599

(22) PCT Filed: Nov. 17, 2003
(86) PCT No.: PCT/EP03/12857

§ 371 Date: May 19, 2005

(30) Foreign Application Priority Data
Nov. 19, 2002 (DE) 10254481.6

(51) Int. Cl. A61N 1/39

(52) U.S. Cl. 607/5; 607/7

(57) ABSTRACT

A defibrillator including an output stage, which is controlled by a control circuit for emitting a biphasic defibrillation pulse and which has an H-bridge between a positive pole and a negative pole of an energy accumulator device. A patient electric circuit is provided in a shunt arm having at least one inductive resistor. This invention achieves a reliable functioning of the defibrillator and thus to regulate the patient current in one direction, only the switching element, which is assigned to this current direction and which is located in the H-limb that points toward the negative pole, is controlled with the higher frequency, whereas in order to regulate the patient current in the other direction, only the switching element, which is assigned to this other current direction and which is located in the H-limb that points toward the positive pole, is controlled with the higher frequency. This invention provides at least one diode that is arranged in an antiparallel manner with regard to the switching elements, which are controlled with the higher frequency, so that via each diode and the switching element, which is constantly closed in the respective phase, the patient current is maintained in its respective direction.

### BACKGROUND OF THE INVENTION

1. Field of the Invention

This invention relates to a defibrillator having an output stage which has an H-bridge between a positive pole and a negative pole of an energy storage unit, and which is triggered via a trigger circuit to emit a bi-phased defibrillation pulse, wherein a patient circuit is embodied in the transverse branch having at least one inductive resistor, and the bi-phased triggering takes place in a manner known per se by alternatively switching on switching members arranged in the four H-legs of the H-bridge for reversing the direction of the patient current in the transverse branch, and wherein the patient current is controlled during the various phases by presetting a reference variable and including an actual value by the trigger circuit by triggering the switching member arrangement with a higher frequency than that for generating the two opposite phases.

2. Discussion of Related Art

A defibrillator is disclosed by German Patent Reference DE 100 65 104 A1. In connection with this known defibrillator with a controlled output stage for the pulse-like bi-phased charging of electrodes, which are to be applied to a patient, with electrical energy from an energy storage unit, an H-bridge is provided in one embodiment, in which transverse branch a current sensor, an inductive resistor in the form of a coil, and patient electrodes with the patient resistor are arranged in series. A semiconductor switch triggered by a control device is arranged in each of the four H-legs, through which the patient current in the transverse branch can be controlled in two opposite directions by an appropriate triggering, such as later described in greater detail and known per se. Pulses of successive opposite current directions are thus generated for defibrillation in accordance with existing knowledge, and are more easily tolerated by the heart tissue than mono-phased pulses with the same energy. During individual current phases, the current is regulated by comparing an actual value of the patient current with a reference variable, wherein the switch arrangement is triggered at a higher frequency than the frequency of the bi-phased pulse in a manner described in greater detail. At the high pulse voltages of, for example, 1 kV and more, and the high frequencies of, for example, more than 10 kHz for the current regulation of the bi-phased pulses of, for example, a few 100 Hz, it is difficult to control the switching members triggered at the higher frequencies in a suitable manner, so that errors and malfunctions of the output stage are not impossible.

### SUMMARY OF THE INVENTION

One object of this invention is to provide a defibrillator of the type mentioned but in which a high degree of dependability of the emission of bi-phased defibrillation signals is achieved.

This object is attained by characteristics described in this specification and in the claims. For regulating the patient current in the one direction, only the switching member assigned to this current direction in the H-leg pointing to the negative pole is triggered at the higher frequency, while for regulating the patient current in the other direction only the switching member assigned to this other current direction in the H-leg pointing to the positive pole is triggered at the higher frequency. Anti-parallel with the switching members triggered at the higher frequency, at least one diode is respectively arranged, so that via the latter, as well as via the switching member continuously closed in the respective phase, the patient current is maintained in its respective direction even if the switching member triggered at the higher frequency is in the open state.

For current regulation in the two phases with opposite current directions, it is always assured when opening the switching members triggered at the higher frequency with these steps, that the patient current is maintained in the direction provided in the respective phase in the transverse branch, wherein the circuit is maintained in the manner of a free-wheeling circuit via the at least one diode, which is anti-parallel with respect to the continuously open switching member, and via the switching member which is continuously closed during the respective phase. For all practical purposes, the current regulation can be performed in accordance with any arbitrary standards via the switching member triggered at the higher frequency and can also be adjusted, for example, in accordance with a changing reference variable standard. A dependable operation of the switching members, and therefore of the output stage, as well as of the entire defibrillator, is assured even at high trigger frequencies of, for example, about 10 or 100 kHz, and at the required high voltages of, for example, on the order of magnitude of 1 kV or higher.

Dependable operation, along with a simple construction is favored because a current sensor resistor is arranged in the transverse branch for detecting the patient current. A proportional voltage is formed from the patient current, which is amplified by an amplifier and has the form of an actual value for a comparison between an internal reference voltage and an external reference voltage. If the external reference voltage is exceeded, a trigger signal of the higher frequency is formed for opening the respective switching member and if the interior reference voltage is downwardly exceeded, a trigger signal of the higher frequency for closing the respective switching member is formed.

Thus, a simple, dependable embodiment results if the trigger signal of the higher frequency is formed via a logic circuit. A programmable micro-controller or a circuit network with memory members and a logic module, for example, can be considered for the logic circuit.

Those steps, wherein the amplified proportional voltage is rectified before, during or after the amplification, contribute to the accuracy of the regulation.

The dependable functioning of the output stage is aided because at a connecting point in the transverse branch between a patient resistor and the inductive resistor placed in series therewith, a respective further diode arrangement in regard to the energy storage unit is respectively arranged both in the blocking direction in the direction toward the positive pole and also in the direction toward the negative pole. Thus, high transient voltage pulses in a downstream located coupling circuit with, for example, a coupling relay, are suppressed. Therefore these further diode arrangements do not constitute a portion of the actual H-bridge.

If, in addition, the two switching members in the two remaining H-legs are bridged by anti-parallel arranged diodes, negative peaks in the remaining switching members which determine the current direction in the two different phases are suppressed.

### BRIEF DESCRIPTION OF THE DRAWINGS

This invention is explained in greater detail in view of an exemplary embodiment making reference to the drawings, wherein:

FIG. 1 shows an output stage and a trigger circuit of a defibrillator connected to it, in a schematic representation;

FIG. 2 is a schematic representation showing the functioning of the output stage in one operating state; and

FIG. 3 is a schematic representation of the output stage in another operating state.

### DESCRIPTION OF PREFERRED EMBODIMENTS

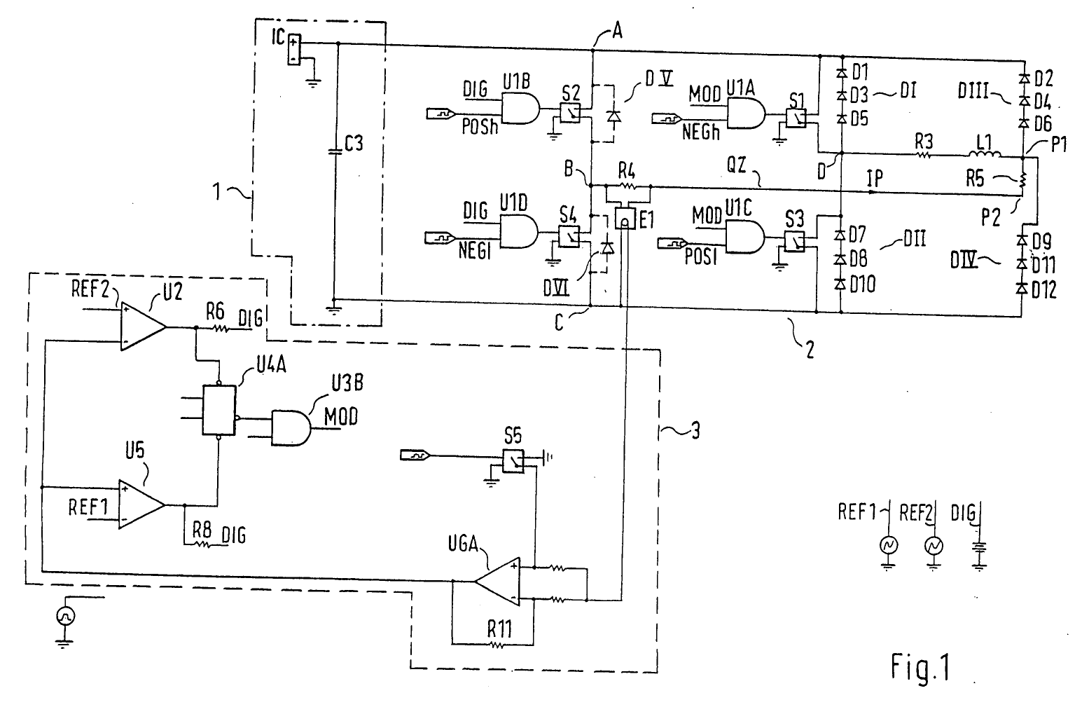

FIG. 1 shows essential components of a portable, externally usable defibrillator in particular, an output stage with an energy storage unit 1 and a high voltage element with the H-bridge 2, as well as a trigger circuit 3 connected to the output stage.

In a customary way, for example as described in German Patent Reference DE 100 65 104 A1, the energy storage unit 1 can have a charging device IC and an energy storage unit C3 connected thereto, such as a capacitor arrangement with at least one capacitor, or an accumulator. The H-bridge 2 connected to the energy storage unit 1 is designed for generating bi-phased defibrillation pulses, for example of a frequency of some 100 Hz, wherein the voltage of the pulses can be on an order of magnitude of 1 kV or higher, such as is also known per se from German Patent Reference DE 100 65 104 A1. In this case the bi-phased pulses are generated in that by triggering switching members, in particular semiconductor switching members S1, S2, S3, S4, such as for example IGBTs, arranged in the H-legs, a patient current IP is generated in two opposite directions in the transverse branch QZ during the two phases of the defibrillation pulse. The patient electrodes to be connected to the patient, or a corresponding selectively connectable patient resistor R5, for example for testing, are arranged in the transverse branch QZ in series with a circuit element forming an inductive resistor L1, in particular a coil or an equivalent component. Also, a sensor resistor R4 for picking up a value of the patient current, is located in the transverse branch QZ in series with the patient resistor R5, or the connected patient. A different sensor element is also possible. In the present embodiment a further series-connected resistor R3 is provided in the transverse branch QZ.

The H-legs pointing toward the positive pole of the energy storage unit 1 therefore extend between a circuit point A on the side of the positive pole, and circuit points B and D on the side of the transverse branch QZ, while the H-legs pointing to the negative pole of the energy storage unit 1 extend from the circuit points B and D to the circuit point C of the negative pole (ground) of the energy storage unit 1. The patient resistor R5, or the patient electrodes instead, are located between connecting points P1, P2 of the transverse branch QZ. The switching members S1, S2, S3, S4 are triggered via respective trigger members U1A, U1B, U1C or U1D by trigger signals from the trigger circuit 3.

Between the circuit points A and D, namely anti-parallel with respect to the switching member S1 on the one side, and between the circuit points D and C, namely anti-parallel in respect to the switching member S3 on the other side, diode arrangements DI and DII are respectively located, which in this case are composed of several diodes D1, D3, D5, or D7, D8, D10. With of the anti-parallel connection, the diode arrangements DI and DII therefore block the current flow from the positive pole to the negative pole of the energy storage unit 1, so that with open switching members S1, S2, S3, S4 no current can flow. Additional diode arrangements DIII and DIV are arranged in a corresponding anti-parallel connection between the connecting point P1 and the switching point A, as well as the switching point C, wherein in the present case the diode arrangements DIII and DIV also have several diodes D2, D4, D6, or D9, D11, D12.

The trigger member U1B of the switching members S2 between the circuit points A, B is assigned control signal inputs for signals DIG and POSh, while control signal inputs for control signals DIG and NEGI are assigned to the trigger member U1D. Control signal inputs for control signals MOD, NEGh are assigned to the trigger member U1A of the switching member S1 between the circuit points A and D, while control signal inputs for control signals MOD and POSI are assigned to the trigger member U1C for the switching member S3 between the circuit points D, C. Thus the four switching members can be suitably triggered for forming the bi-phased pulses and for current regulation during the respective phases of opposite current direction in the transverse branch QZ, wherein the control signals DIG and MOD are formed and supplied by a current regulating arrangement of the trigger circuit 3.

The current control arrangement of the trigger circuit 3 has a current feedback branch, which picks up a proportional voltage E1 via the sensor resistor R4 and provides it to an amplifier with a circuit element U6A. The voltage provided the circuit element U6A, or the amplifier connected with it, are rectified by a change-over switch S5, so that the voltage at the output of the amplifier with the circuit element U6A has the same polarity in both phases and is applied in the same way to comparator units U2, U5 and can be compared with given values REF2 or REF1. In this case the given values represent an interior reference voltage REF1, or an exterior reference voltage REF2, by which a regulating interval for the desired current within an hysteresis band is formed. A reference variable/actual value comparison thus takes place in both phases in a corresponding manner via the comparator units U2, U5. The comparison results are provided to a memory member U4A, for example a bi-stable memory member or trigger member, to one output of which a logic member U3B is connected, which has a further control input. The outputs of the comparator units U2, U5 make the control signal DIG available via the resistors R6 or R8, while the control signal MOD is present at the output of the logic member U3B.

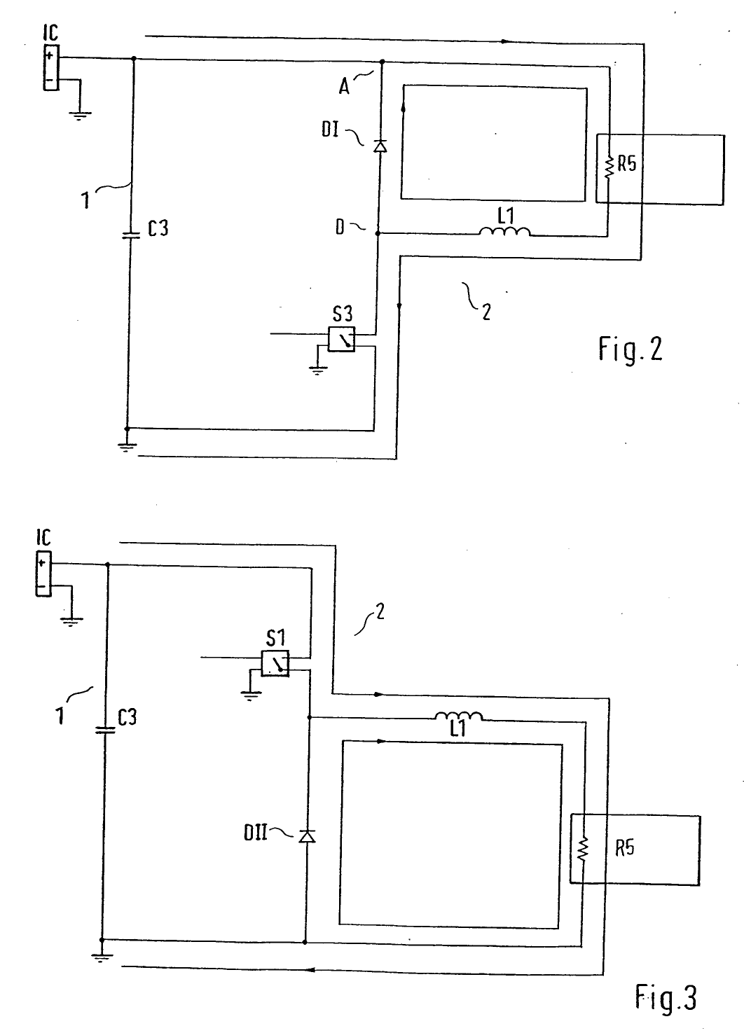

For current regulation in the one phase, the switching member S2 between the circuit points A and B is continuously closed, while the switching member S3 between the circuit points D and C is triggered with a higher frequency of the control signal for regulating the current. The two other switching members S1 between the circuit points A and D, and S4 between the circuit points B and C are continuously open in this phase. This situation is represented in FIG. 2, wherein only the switching member S3 triggered with the higher frequency in this phase is represented, because the switching member S2 is continuously closed. If the switching member S3 is closed, the patient current flows through the patient, or the patient resistor R5, from the positive pole to the negative pole of the energy storage unit 1. If the switching member S3 is open, the patient current flows through the transverse branch QZ in the same direction, but through the first diode arrangement DI in the H-leg from the circuit point D to A, so that a free-wheeling circuit is formed. In this case, triggering of the switching member S3 takes place in accordance with the current regulation, wherein, in the closed state of the switching member S3, the current rises from the energy storage unit C3 through the resistor R5, or the impedance of the patient, the inductive resistor L1 and the switching member S3 until the threshold value formed by the exterior reference voltage REF2 is reached. If then the switching member S3 is opened, the patient current drops through the patient resistor R5, or the patient, and the inductive resistor L1 as well as the diode arrangement DI, until the reference value formed by the interior reference voltage REF1 is reached, after which the switching member S3 is closed again. These regulating actions are repeated until the respective phase of the patient current IP is finished, which for example lasts some, or some tenths of a millisecond.

For causing the phase with the opposite patient current IP in the transverse branch QZ, the switching members S2, S3 are opened, while the switching member S4 between the circuit points B and C remains constantly closed, and the switching member S1 between the circuit points D and A is triggered with a higher frequency for regulating the current. This situation is represented in FIG. 3, wherein only the switching member S1, which is relevant, is drawn in. When the switching member S1 is in the switched-on state, the patient current IP flows from the energy storage device C3 through the switching member S1, the inductive resistor L1 and the patient resistor R5, or the patient, to ground. If in this state the switching member S1 is opened, the patient current IP continues to flow in a corresponding direction through the patient resistor R5, or the patient, the inductive resistor L1, as well as the diode arrangement DII, so that a free-wheeling circuit is also formed. This state continues until the value of the patient current IP corresponding to the interior reference voltage REF1 is reached, after which the switching member S1 is closed again by the current regulating circuit, and so forth, until this opposite phase of the patient current IP, or of the defibrillation pulse, is terminated. The different pulse phases can be alternatingly repeated with a suitably frequency.

By specifying appropriate reference values REF1, REF2, the current regulation can be affected in the desired way, such as by determination of the width and shape of the hysteresis band, wherein a dependable operation of the defibrillator can be achieved by the described design of the H-bridge and its triggering.

The switching members S2 or S4 in the H-legs A-B, or B-C, which are continuously switched on in the respective phases, can also be bridged by diodes DV or DVI in an anti-parallel arrangement, by which negative voltage excesses in these switching members S2 or S4 can be suppressed.

1. A defibrillator having an output stage with an H-bridge (2) between a positive pole and a negative pole of an energy storage unit (1), and which is triggered via a trigger circuit (3) to emit a bi-phased defibrillation pulse, wherein a patient circuit is in the transverse branch (QZ) having at least one inductive resistor (L1), and the bi-phased triggering takes place by alternatively switching on switching members (S1, S2, S3, S4) arranged in four H-legs of the H-bridge (2) for reversing a direction of the patient current (IP) in the transverse branch (QZ), and wherein the patient current (IP) is controlled during the various phases by presetting a reference variable and including an actual value by the trigger circuit (3) by triggering the switching members (S1, S2, S3, S4) with a higher frequency than that for generating the two opposite phases, the defibrillator comprising:
for regulating the patient current (IP) in the one direction, only the switching member (S3) assigned to the current direction in the H-leg (D-c) pointing to the negative pole triggered at the higher frequency, while for regulating the patient current (IP) in an other direction only the switching member (S1) assigned to the other current direction in the H-leg (D-A) pointing to the positive pole is triggered at the higher frequency; and
anti-parallel with the switching members (S3, S1) triggered at the higher frequency, at least one diode (DII, DI) is respectively arranged with the switching member (S2, S4) continuously closed in the respective phase to maintain the patient current (IP) in a respective direction even if the switching member (S1, S3) triggered at the higher frequency is in an open state.

2. The defibrillator in accordance with claim 1, wherein a current sensor resistor (R4) is arranged in the transverse branch (QZ) for detecting the patient current (IP), a proportional voltage (E1) is formed from the patient current (IP) which is amplified by an amplifier (U6A) and is in a form of an actual value for a comparison between an internal reference voltage (REF1) and an external reference voltage (REF2), and if the external reference voltage (REF2) is exceeded a trigger signal of the higher frequency is formed for opening the respective switching member (S3, S1), and if the interior reference voltage (REF1) is downwardly exceeded, a trigger signal of the higher frequency for closing the respective switching member (S3, S1) is formed.

3. The defibrillator in accordance with claim 2, wherein the trigger signal of the higher frequency is formed via a logic circuit (U4A, U3b).

4. The defibrillator in accordance with claim 3, wherein the amplified proportional voltage (E1) is rectified one of before, during and after the amplification.

5. The defibrillator in accordance with claim 4, wherein at a connecting point (P1) in the transverse branch (QZ) between a patient resistor (R5) and the inductive resistor (L1) placed in series therewith, a further diode arrangement (DIII, DIV) regarding the energy storage unit is respectively arranged in the blocking direction in a positive direction toward the positive pole and in a negative direction toward the negative pole.

6. The defibrillator in accordance with claim 5, wherein the two switching members (S2, S4) in the two remaining H-legs (A-B, B-C) are bridged by anti-parallel arranged diodes (DV, DVI).

7. The defibrillator in accordance with claim 2, wherein an amplified proportional voltage (E1) is rectified one of before, during and after the amplification.

8. The defibrillator in accordance with claim 1, wherein at a connecting point (P1) in the transverse branch (QZ) between a patient resistor (R5) and the inductive resistor (L1) placed in series therewith, a further diode arrangement (DIII, DIV) regarding the energy storage unit is respectively arranged in the blocking direction in a positive direction toward the positive pole and in a negative direction toward the negative pole.

9. The defibrillator in accordance with claim 1, wherein the two switching members (S2, S4) in the two remaining H-legs (A-B, B-C) are bridged by anti-parallel arranged diodes (DV, DVI).
